(19) United States
(12) Patent Application Publication
(10) Pub. No.: US 2015/0366998 A1
(43) Pub. Date: Dec. 24, 2015

(54) ORAL DELIVERY OF DRUG ACTIVES IN LABORATORY ANIMALS USING FAST-DISSOLVING ORAL FILMS

(71) Applicants: Eric Allen, Camarillo, CA (US); Ed Maliski, Thousand Oaks, CA (US); Daryl Davies, San Pedro, CA (US)

(72) Inventors: Eric Allen, Camarillo, CA (US); Ed Maliski, Thousand Oaks, CA (US); Daryl Davies, San Pedro, CA (US)

(21) Appl. No.: 14/747,698

(22) Filed: Jun. 23, 2015

Related U.S. Application Data
(60) Provisional application No. 62/015,701, filed on Jun. 23, 2014.

(51) Int. Cl. A61K 49/00

(52) U.S. Cl. CPC A61K 49/0008

(57) ABSTRACT

The invention provides a method of orally delivering a pharmaceutical composition to a test animal, and a method of evaluating for treatment or diagnosis of a medical condition, a pharmaceutical composition administered orally to a test animal, including administering to the test animal an effective amount of an oral thin film including the pharmaceutical composition, wherein orally delivering a pharmaceutical composition to the test animal is performed to evaluate the safety and efficacy of the pharmaceutical composition, and wherein reduced trauma is associated with administration of the pharmaceutical composition by oral thin film to the test animal, compared to a gavage administration. Further, a method of treatment of an alcohol uptake disorder (AUD) in a patient is provided, including administering to the patient afflicted therewith an effective amount of an oral thin film formulation of ivermectin.

### PRIORITY APPLICATION

This application claims the benefit under 35 U.S.C. 119(e) to U.S. Provisional Application No. 62/015,701, filed on 23 Jun. 2014; which application is incorporated herein by reference in its entirety.

### BACKGROUND

Alcohol use disorders (AUDs) rank third on the list of preventable causes of morbidity and mortality in the United States, affecting over 18 million people and causing over 100,000 deaths annually. The economic burden to society for AUDs is in excess of $220 billion annually which exceeds the costs of other leading preventable causes of death such as cigarette smoking and physical inactivity. Unfortunately, current pharmacologic and other therapeutic approaches have had limited success.

To address this problem, our laboratory is developing a new pharmacological treatment strategy for AUDs based on our demonstration that ivermectin (IVM), an FDA approved anthelmintic agent, can significantly reduce ethanol intake in mice following acute and 7 day administration (Yardley 2012). Further studies revealed an inverse relationship between the concentrations of IVM in the brain and the degree of ethanol intake in mice (Yardley 2012). Moreover, IVM, at doses needed to produce the anti-alcohol effects, elicited anxiolytic-like effects, but did not induce overt signs of toxicity, which is a significant problem that produces a high level of non-compliance with current therapies (Bortolato 2013). Importantly, this IVM regimen did not exert rewarding properties indicating that the IVM does not have addiction liability. Collectively, our initial findings point to the potential of repurposing IVM as novel treatment of AUDs.

To further explore the potential of IVM as a therapy for AUDs, the current study tested IVM efficacy in mice in a 30 day study delivering IVM via fast dissolving oral films. The findings indicate that IVM significantly reduces alcohol intake and preference over 30 days without significantly affecting fluid intake, body weight, or organ weight to tibia ratio compared to the placebo film group and the no film group. This first demonstration that orally administered IVM can reduce ethanol intake over a prolonged period without causing overt signs of toxicity supports translation into clinical trials of IVM for the prevention and/or treatment of AUDs.

Pharmaceutical compositions, such as IVM, can be administered to an animal orally for purposes including evaluation of the pharmaceutical composition, such as for obtaining pharmacokinetic data, such as ADMET data, and the like. It is important that animals used for such evaluations provide reliable and reproducible models for evaluation the use or potential for use of the pharmaceutical composition in medical treatments of human patients.

It is well known that oral gavage, commonly used as a method of oral administration of test substances like pharmaceutical compositions to animals for toxicity and metabolism studies, is traumatic to animals experiencing the procedure. Insertion of the feeding tube into the animal can produce physical injury, and mental trauma is also reported by human subjects who have undergone the procedure.

### SUMMARY

In various embodiments, the present invention provides for a method of orally delivering a pharmaceutical composition to a test animal (e.g., rabbit or mouse). The method includes administering to the test animal an effective amount of an oral thin film that includes the pharmaceutical composition. The method of orally delivering the pharmaceutical composition to the test animal is performed to evaluate the safety and efficacy of the pharmaceutical composition. In various embodiments, advantages of the methods of the invention include reduced trauma associated with administration of the pharmaceutical composition by oral thin film to the test animal, compared to a gavage administration. In various embodiments, advantages of the methods of the invention include obtaining data from the test animal that is more accurate, precise and/or reliable, compared to data obtained with a gavage administration.

It requires much less skilled effort by the technician to deliver using the thin strip, compared to oral gavage. Oral gavage is time consuming, labor intensive and requires a much higher level of skilled personnel. The thin strip is highly advantageous because of these points. Oral gavage may cause trauma when doing any type of behavioral study, i.e., to answer the question does drug X alter some behavior or treat some condition. The data for oral gavage can be skewed due to the gavaging. This would be especially true if one considers a long term study, gavaging the animal every day, which would become very traumatic for the animal. The gavage method can induce stress—which can change how the “body” affects the drug, per se.

### BRIEF DESCRIPTION OF THE FIGURES

FIG. 1 is (left) a graph showing a time course of treatment with ivermectin (IVM) administered via an oral thin film (OTF) vehicle, the y-axis indicating mass of consumption by test animals of a 10% ethanol in water solution (10E) of IVM/OTF treated animals, versus placebo OTF and no action (N/A) regimens; (right) a bar graph summary of the data. In this study, IVM was given for 30 days, 5 times per week for 6 weeks. There were 4 groups as described below. The IVM group drank consistently less 10E over the 30-day period compared to the N/A control and placebo group. IVM (0.21 mg), significantly decreases alcohol intake in a 24-h two bottle choice paradigm. Treatment with IVM (0.21 mg) delivered via fast dissolving oral film significantly decreased 10E intake by approximately 40% compared to baseline (n=8). There is no different between baseline 10E intake (black bar; n=22), no treatment (white bar; n=6), and placebo-key lime flavored fast dissolving oral films (light grey bar, n=8). Values represent mean±SEM for female WT mice. ***p<0.001 vs baseline.

FIG. 2 and FIG. 3 show that IVM/OTF administration significantly decreased 10E preference and increased water intake, and FIG. 4 shows that total fluid intake was substantially unchanged.

FIG. 5 shows that animal weight gain in IVM/OTF treatment versus no action was substantially unaffected, while FIG. 6 shows a significant increase in food intake with the IVM/OTF treated group.

FIG. 7 shows that IVM/OTF administration had substantially no effect on organ weight or tibia length ratio.

FIG. 8 shows that 30-day administration of IVM/OTF (0.21 mg level) decreased 10E intake, while there was no significant difference between no action and placebo treatments and baseline in terms of 10E intake.

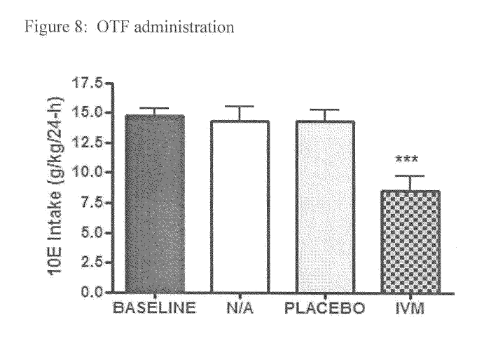

FIG. 9 shows that oral gavage administration of IVM (5 mg/kg, gavage) decreased 10E intake, while there was a very significant difference in 10E intake between no action and placebo gavage (corn oil), indicating a result of animal trauma from the gavage procedure.

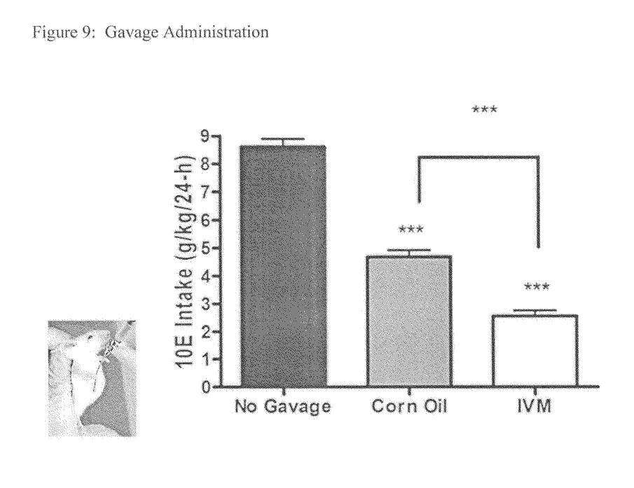

### DETAILED DESCRIPTION

The present invention is directed, in various embodiments, to a method of orally delivering a pharmaceutical composition to a test animal, including administering to the test animal an effective amount of an oral thin film including the pharmaceutical composition, wherein orally delivering a pharmaceutical composition to the test animal is performed to evaluate the safety and efficacy of the pharmaceutical composition, and wherein reduced trauma is associated with administration of the pharmaceutical composition by oral thin film to the test animal, compared to a gavage administration. The data thus obtained from the test animal can be more accurate, precise and/or reliable, compared to data obtained with a gavage administration.

The traumatic effects of gavage administration, where a liquid or semi-solid composition is force-fed via a tube or syringe needle directly through the mouth or nose of an animal to the animal's esophagus and stomach, are well known. Numerous descriptions of the traumatic effects of gavage as administered to humans, e.g., in jails and the like, support the understanding that gavage is a traumatic procedure to the recipient. This is also true of gavage administration to animals; force-feeding of geese to yield the enlarged goose liver of foie gras has been criticized by animal welfare groups as cruel and painful treatment for the animals.

In evaluation of the safety and efficacy of a pharmaceutical composition, e.g., including a bioactive agent, it is necessary to orally administer the composition to an animal, such as a rodent, to study the pharmacokinetics, toxicity, and the like such as is carried out in an ADMET study in support of registration of a drug for human use. It is preferred for animal studies using test animals such as rodents to be carried out under reliable and reproducible conditions, and for the animals to be in as good and uniform a condition as possible for the study. The trauma associated with oral gavage administration of the test composition can negatively impact the condition of the test animals, and thus effect the accuracy, precision and/or reliability of the data obtained from the stressed, traumatized animal, compared to data obtained using a method of the invention including oral administration of the pharmaceutical composition as an oral thin film (OTF) as disclosed and claimed herein.

An oral thin film as can be used in practice of a method of the invention can be as described in U.S. Ser. Nos. 10/921,770 and 13/890,875, by certain of Applicants herein, which are incorporated by reference herein in their entireties.

In practice of a method of the invention, when the test animal is a rodent, the rodent can exhibit flavor preferences, and will more readily orally ingest an oral thin film when the oral thin film is flavored. Various flavors are available, e.g., mint, vanilla, and other scents such as cheese, meat, and the like, than can induce rodent feeding behavior. In various embodiments of an inventive method, an oral thin film of a first flavor can be preferentially selected by a test animal over an oral thin film of a second flavor.

Thus, the invention can provide a method of evaluating for treatment or diagnosis of a medical condition a pharmaceutical composition administered orally to a test animal, including administering the pharmaceutical composition in an oral thin film formulation to the test animal, wherein reduced trauma is associated with administration of the pharmaceutical composition by oral thin film to the test animal, compared to a gavage administration. Use of medicament-containing oral thin films in carrying out animal (e.g., rodent) testing in support of registration for human use of the medicament is preferred to administration of the pharmaceutical by oral gavage, as described above, because data obtained from the test animal using OTF administration of the pharmaceutical can be more accurate, precise and/or reliable, compared to data obtained with a gavage administration. The reduction of trauma to the animal from use of the OTF compared to gavage administration provides more reliable results. Furthermore, the use of OTF oral administration is preferred on the basis of minimizing pain and discomfort to animals in carrying out necessary studies. The use of flavored oral thin films can further enhance animal comfort during the course of the studies by providing a positive mental reward to the animal upon oral ingestion of the flavored oral thin film, the flavor of which can be selected to increase the animal's appreciation upon oral ingestion.

Furthermore, the present invention provides, in various embodiments, a method evaluation of pharmaceutical compositions for treatment of an alcohol uptake disorder (AUD) including administering to a test animal an effective amount of an oral thin film formulation of the pharmaceutical composition. For instance, the pharmaceutical composition can be ivermectin (IVM), as the exemplary data obtained using ivermectin in the subject matter presented herein illustrates. For example, in various embodiments, the pharmaceutical composition has not received approval from a relevant regulatory authority, such as from the US Food and Drug Agency (US FDA).

The data gathered in the exemplary studies presented herein and described in the Examples and Figures further indicate that the invention provides, in various embodiments, a method of treatment of an alcohol uptake disorder (AUD) in a patient, including administering to the patient afflicted therewith an effective amount of an oral thin film formulation of ivermectin.

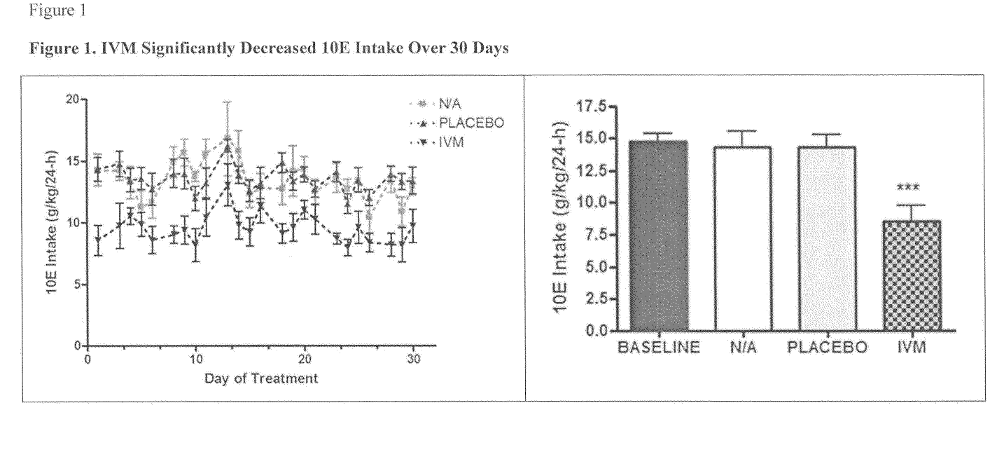

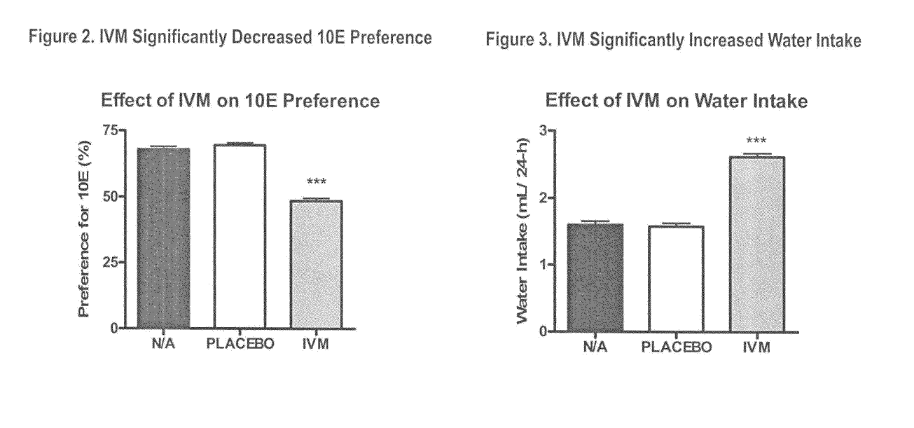

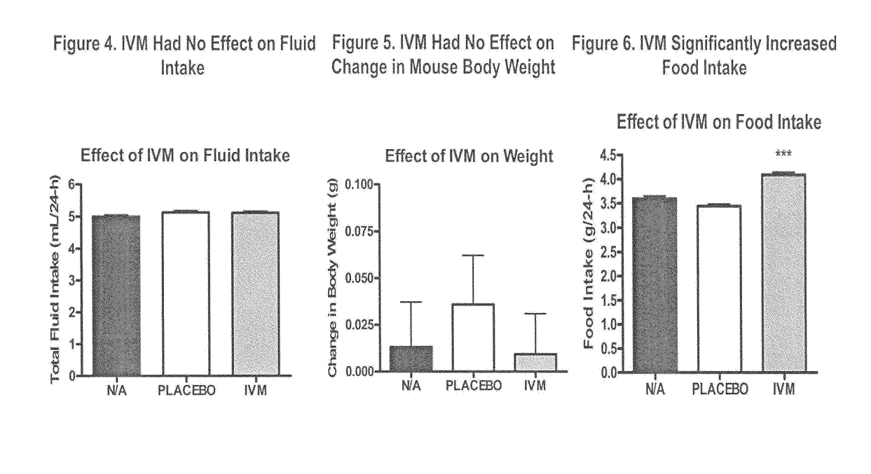

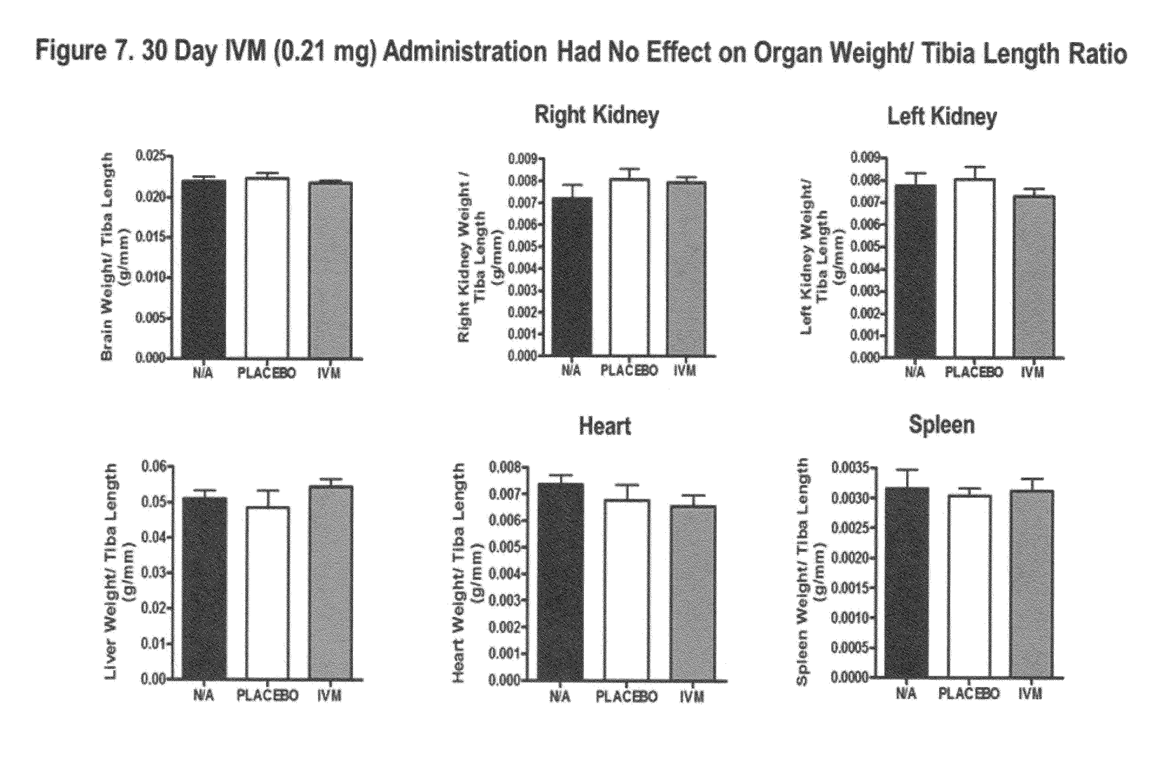

As illustrated in the Figures, using an oral thin film formulation of ivermectin in the study of a potential therapeutic medicament for treatment an alcohol uptake disorder, it is apparent that the ivermectin/oral thin film (IVM/OTF) treated mice drank consistently less 10% ethanol in water (10E) over the 30-day period compared to the control and placebo groups (FIG. 1); furthermore, using the OTF formulation, there was little to no difference between the control (no action)and placebo (OTF film with no medicament) treatments, such as could occur in the case of trauma to the animal. Thus, FIG. 1 indicates that oral administration of a pharmaceutical such as IVM by the oral thin film formulation is effective, and that the result is not biased by the method of oral administration, as those two groups of test animals showed little or no difference in 10E intake. FIG. 2 indicates that the preference for 10E was also significantly less in the IVM group compared to control and placebo groups, while, perhaps to account for the decrease in fluid intake from the 10E bottle, the IVM group drank significantly more water (FIG. 3) but overall, the fluid intake among the 3 groups was similar (FIG. 4). The comparatively neutral effects of OTF administration is further indicated by the observation that the change in body weight was similar across the 3 groups (FIG. 5); however; the IVM group consumed significantly more food, possibly in an attempt to counteract the caloric loss resulting from the decreased 10E consumption (FIG. 6). Additionally, as a result of this study, it was determined that for each of the 6 organs analyzed from the test rodents, there was no significant difference in organ weight to tibia length ratio between the 3 groups (FIG. 7). Histological analyses are ongoing.

AUDs are chronic diseases and a single dose of IVM would normally be insufficient to successfully treat these disorders, therefore, we tested the effect of long-term oral IVM administration on ethanol intake. Our results suggest that chronic oral administration of IVM is effective in reducing 10E intake and is well tolerated.

Taken together, our data support the use of fast dissolving oral films as an effective form of oral drug delivery in mice and also supports the use of IVM as a therapeutic agent for the prevention and/or treatment of AUDs.

### Enumerated Embodiments

Specific enumerated embodiments [1] to [19] provided below are for illustration purposes only, and do not otherwise limit the scope of the invention, as defined by the claims. These enumerated embodiments encompass all combinations, sub-combinations, and multiply referenced (e.g., multiply dependent) combinations described therein.

[1.] A method including administering to a test animal an effective amount of an oral thin film including the pharmaceutical composition, wherein orally delivering the pharmaceutical composition to the test animal is performed to evaluate at least one of safety and efficacy of the pharmaceutical composition.

[2.] A method of evaluating at least one of safety and efficacy of a pharmaceutical composition, the method including orally delivering an oral thin film including the pharmaceutical composition to a test animal.

[3.] A method of evaluating at least one of safety and efficacy of a pharmaceutical composition for least one of treatment and diagnosis of a medical condition, the method including orally delivering an oral thin film including the pharmaceutical composition to a test animal.

[4.] The method of any one of embodiments [1]-[3], wherein the administration occurs in an amount and for a period of time effective to evaluate at least one of the safety and the efficacy of the pharmaceutical composition

[5.] The method of any one of embodiments [1]-[3], wherein the pharmaceutical composition includes an active pharmaceutical ingredient (API).

[6.] The method of any one of embodiments [1]-[5], wherein the pharmaceutical composition has not received approval from a relevant regulatory authority.

[7.] The method of any one of embodiments [1]-[5], wherein the pharmaceutical composition is in clinical trials.

[8.] The method of any one of embodiments [1]-[5], wherein the pharmaceutical composition has not received approval from the U.S. Food and Drug Agency (U.S. FDA).

[9.] The method of any one of embodiments [1]-[8], wherein the evaluating at least one of the safety and the efficacy of the pharmaceutical composition is carried out to evaluate the pharmaceutical composition for at least one of treatment and diagnosis of a medical condition.

[10.] The method of any one of embodiments [1]-[9], wherein reduced trauma is associated with administration of the pharmaceutical composition by oral thin film to the test animal, compared to a gavage administration to the test animal.

[11.] The method of any one of embodiments [1]-[10], wherein data obtained from the test animal is at least one of more accurate, more precise, and more reliable, compared to data obtained with a gavage administration to the test animal.

[12.] The method of any one of embodiments [1]-[11], wherein the test animal is a mammal.

[13.] The method of any one of embodiments [1]-[12], wherein the test animal is a rodent.

[14.] The method of any one of embodiments [1]-[13], wherein the oral thin film includes one or more flavoring agents.

[15.] The method of any one of embodiments [1]-[14], wherein the oral thin film includes one or more flavoring agents selected for the test animal.

[16.] The method of any one of embodiments [1]-[15], wherein after the administration of the pharmaceutical composition, pharmacological or pharmacokinetic data is obtained from the test animal.

[17.] The method of any one of embodiments [1]-[15], wherein after the administration of the pharmaceutical composition, pharmacological or pharmacokinetic data is obtained from the test animal, wherein the pharmacological or pharmacokinetic data includes at least one of:
peak plasma concentration of a drug after administration (Cₘₐₓ),
time to reach Cₘₐₓ (tₘₐₓ),
lowest concentration that a drug reaches before the next dose is administered (C_{min,SS}),
amount of drug in a given volume of plasma (C₀),
time required for the concentration of the drug to reach half of its original value (t_{1/2}),
rate of infusion required to balance elimination (kᵢₙ),
integral of the concentration-time curve (after a single dose or in steady state) (AUC),
volume of plasma cleared of the drug per unit time (CL), and
systemically available fraction of a drug (f).

[18.] A method of evaluating at least one of safety and efficacy of a pharmaceutical composition, the method including orally delivering an oral thin film including the pharmaceutical composition to a rodent,
wherein the administration occurs in an amount and for a period of time effective to evaluate at least one of the safety and the efficacy of the pharmaceutical composition,
wherein after the administration of the pharmaceutical composition, pharmacological or pharmacokinetic data is obtained from the test animal, wherein the pharmacological or pharmacokinetic data includes at least one of:
peak plasma concentration of a drug after administration (Cₘₐₓ),
time to reach Cₘₐₓ (tₘₐₓ),
lowest concentration that a drug reaches before the next dose is administered (C_{min,SS}),
amount of drug in a given volume of plasma (C₀),
time required for the concentration of the drug to reach half of its original value (t_{1/2}),
rate of infusion required to balance elimination (kᵢₙ),
integral of the concentration-time curve (after a single dose or in steady state) (AUC),
volume of plasma cleared of the drug per unit time (CL), and
systemically available fraction of a drug (f).

[19.] A method of treatment of an alcohol uptake disorder (AUD) in a patient, the method including administering to the patient afflicted therewith an oral thin film formulation including an effective amount of ivermectin.

### EXAMPLES

### 24-h Two-Bottle Choice Paradigm

The 24-h two bottle choice paradigm was performed as previously described (Yardley 2012). In this study, there were 4 groups. There is no different between baseline 10E intake (black bar; n=22), no treatment (white bar; n=6), and placebo-key lime flavored fast dissolving oral films (light grey bar, n=8) compared to 4) key lime flavored oral film with IVM (0.21 mg).

### Oral Film Administration

IVM was given for 30 days, 5 times per week for 6 weeks. IVM was administered using an animal feeding needle dipped in a 4.25% sucrose solution to serve as an adhesive for the films strips. After being presented with the tip of the gavage needle, mice would self-administer the oral films.

### Data Analysis

Ethanol intake was expressed as g/kg/24-h and summarized using means±SEM across groups. A two-way ANOVA was used to analyze 10E intake between the 4 groups over the 30 days of treatment. A one-way ANOVA was used to compare mean 10E intake, 10E preference, water intake, fluid intake, food intake, mouse weight, and organ weight/tibia length ratio across the 4 groups. GraphPAD Prism software (San Diego, Calif.) was used for data analysis.

What is claimed is:

1. A method of evaluating at least one of safety and efficacy of a pharmaceutical composition, the method comprising orally delivering an oral thin film comprising the pharmaceutical composition to a test animal.

2. The method of claim 1, wherein the administration occurs in an amount and for a period of time effective to evaluate at least one of the safety and the efficacy of the pharmaceutical composition.

3. The method of claim 1, wherein the pharmaceutical composition comprises an active pharmaceutical ingredient (API).

4. The method of claim 1, wherein the pharmaceutical composition has not received approval from a relevant regulatory authority.

5. The method of claim 1, wherein the pharmaceutical composition is in clinical trials.

6. The method of claim 1, wherein the pharmaceutical composition has not received approval from the U.S. Food and Drug Agency (U.S. FDA).

7. The method of claim 1, wherein the evaluating at least one of the safety and the efficacy of the pharmaceutical composition is carried out to evaluate the pharmaceutical composition for at least one of treatment and diagnosis of a medical condition.

8. The method of claim 1, wherein reduced trauma is associated with administration of the pharmaceutical composition by oral thin film to the test animal, compared to a gavage administration to the test animal.

9. The method of claim 1, wherein data obtained from the test animal is at least one of more accurate, more precise and more reliable, compared to data obtained with a gavage administration to the test animal.

10. The method of claim 1, wherein the test animal is a mammal.

11. The method of claim 1, wherein the test animal is a rodent.

12. The method of claim 1, wherein the oral thin film comprises one or more flavoring agents.

13. The method of claim 1, wherein the oral thin film comprises one or more flavoring agents selected for the test animal.

14. The method of claim 1, wherein after the administration of the pharmaceutical composition, pharmacological or pharmacokinetic data is obtained from the test animal.

15. The method of claim 1, wherein after the administration of the pharmaceutical composition, pharmacological or pharmacokinetic data is obtained from the test animal, wherein the pharmacological or pharmacokinetic data comprises at least one of:
peak plasma concentration of a drug after administration (Cₘₐₓ),
time to reach Cₘₐₓ (tₘₐₓ),
lowest concentration that a drug reaches before the next dose is administered (C_{min,SS}),
amount of drug in a given volume of plasma (C₀),
time required for the concentration of the drug to reach half of its original value (t_{1/2}),
rate of infusion required to balance elimination (kᵢₙ),
integral of the concentration-time curve (after a single dose or in steady state) (AUC),
volume of plasma cleared of the drug per unit time (CL), and
systemically available fraction of a drug (f).

16. A method of evaluating at least one of safety and efficacy of a pharmaceutical composition, the method comprising orally delivering an oral thin film comprising the pharmaceutical composition to a rodent,
wherein the administration occurs in an amount and for a period of time effective to evaluate at least one of the safety and the efficacy of the pharmaceutical composition,
wherein after the administration of the pharmaceutical composition, pharmacological or pharmacokinetic data is obtained from the rodent, wherein the pharmacological or pharmacokinetic data comprises at least one of:
peak plasma concentration of a drug after administration (Cₘₐₓ),
time to reach Cₘₐₓ (tₘₐₓ),
lowest concentration that a drug reaches before the next dose is administered (C_{min,SS}),
amount of drug in a given volume of plasma (C₀),
time required for the concentration of the drug to reach half of its original value (t_{1/2}),
rate of infusion required to balance elimination (kᵢₙ),
integral of the concentration-time curve (after a single dose or in steady state) (AUC),
volume of plasma cleared of the drug per unit time (CL), and
systemically available fraction of a drug (f).
